(19) United States
(12) Patent Application Publication
(10) Pub. No.: US 2015/0320918 A1
(43) Pub. Date: Nov. 12, 2015

(54) POINT OF CARE ISOLATION AND CONCENTRATION OF BLOOD CELLS

(71) Applicant: BHC TECHNOLOGY HOLDINGS LLC, St. Paul, MN (US)

(72) Inventors: Daniel Patrick COLLINS, Lino Lakes, MN (US); Joel Harry HAPKE, Brooklyn Center, MN (US)

(21) Appl. No.: 14/652,049

(22) PCT Filed: Dec. 13, 2013
(86) PCT No.: PCT/US2013/075056

§ 371 Date: Jun. 12, 2015

Related U.S. Application Data
(60) Provisional application No. 61/737,350, filed on Dec. 14, 2012.

(51) Int. Cl. A61M 1/02; A61B 10/00; A61B 5/15; C12N 5/00; A61M 39/22

(52) U.S. Cl. CPC A61M 1/0259; C12N 5/0087; A61M 39/223; A61B 5/150038; A61B 5/150045; A61B 10/0045; A61B 2010/008

(57) ABSTRACT

The invention provides systems, methods, compositions, and separation media for cell separation and for the concentration of therapeutically important cells from blood cell containing tissues. The systems, methods, reagents and techniques specifically agglutinate cells via surface antigen recognition and can be used to recover even rare cell types in high yield.

### CROSS-REFERENCE TO RELATED APPLICATION

This application claims benefit of U.S. Provisional Application No. 61/737,350, filed on 14 Dec. 2012 and which application is incorporated herein by reference. A claim of priority is made.

### BACKGROUND

Blood cells were the first tissue to be successfully transplanted, in the form of transfusion of red blood cells. Transfusions were the solution to mortality resulting from acute blood loss and have led to the establishment of blood banks worldwide that store blood cells and components for therapeutic applications.

Blood tissues contain a wide variety of cells that have been shown to have therapeutic potential. Bone marrow and umbilical cord blood contain stem cells that are capable of completely restoring a hematopoietic system. Bone marrow and cord blood transplants are the therapy of last resort in the treatment of leukemia and other blood disorders.

Improved methods and systems are needed for enhancing the safety and effectiveness of blood products for therapeutic applications.

### SUMMARY

A system describing compositions, materials and methods for removing undesired cell types from blood tissues and concentrating the resultant cell suspension to user determined volumes are provided herein. The disclosed system and methods can be used, for example, to prepare cells for tissue culture, diagnostic testing, further purification, cryogenic storage, or therapeutic applications. While the system and methods described are useful for many applications, this invention is especially relevant for point of care isolation and concentration of autologous bone marrow osteogenic progenitors for orthopedic applications where bone marrow aspirates are the treatment of choice.

Applicants have invented a system and methods to reduce erythrocytes and inflammatory granulocytes without reduction of the stem cell component in the bone marrow aspirate, which provides an improved, effective and more concentrated therapeutic for orthopedic and other therapeutic applications.

This system is comprised of a series of interconnected syringes, valves, a filter and a cell separation medium. Methods include introducing a cell-containing biological sample (e.g., a peripheral blood sample, umbilical cord blood, or bone marrow aspirate) to a syringe containing the cell separation medium and mixing the sample and the cell separation medium. After mixing, the syringe is placed in an upright position with the plunger side facing down and the sample is allowed to settle and separate into a lower portion containing the erythrocytes and other undesired cells and an upper portion containing the desired cells in suspension. After the settling period, the valves between the syringe and the filter are opened and the cell-containing suspension is passed into the filter chamber by compressing the plunger. After completing the transfer of cell suspension into the filter chamber, the valve to the sample syringe is closed. Compression via the shuttle syringes causes the fluid portion of the cell suspension to pass through the filter, concentrating the cells behind the filter in a smaller volume. After reducing the volume to the desired level, the cell suspension is transferred into a final syringe for further applications.

Provided herein are systems for separating blood tissue and concentrating the desired therapeutic cells comprising a cell concentration device, and an effective amount of a cell separation medium. The cell concentration devices comprise one or more syringes, one or more valves, and a filtration device, wherein the syringes, valves and filtration device are assembled together to allow for the concentration of desired therapeutic cells from said blood tissue. In an embodiment, the filtration device is a tangential flow filtration device. In another embodiment, the cell separation medium comprises an effective amount of a zeta potential reducing agent and an effective amount of a Ca⁺² chelating agent in a buffered solution. In the cell separation media provided herein, the zeta potential reducing agent can be Heta starch, the Ca⁺² chelating agent can be EDTA, and the buffered solution is a phosphate buffered solution.

In some embodiments, the cell separation media of the invention can contain Heta starch at a concentration ranging from 1.0% to 4.0, or a concentration ranging from 1.5% to 3.0%. The cell separation media can contain EDTA at a concentration ranging from 0.05 mM to about 20 mM, or, optionally, a concentration ranging from 0.1 mM to about 10 mM.

In other embodiments, a cell separation medium is provided for the removal of erythrocytes, granulocytes and monocytes from a blood cell containing sample, comprising an effective amount of a zeta potential reducing agent, an effective amount of Ca⁺² ions, an effective amount of Mg⁺² ions, and an effective amount of an anti-CD15 antibody, where the zeta potential reducing agent, the Ca⁺² ions, the Mg⁺² ions, and the anti-CD15 antibody are contained in a buffered physiologic saline solution. In certain aspects, the compositions contain Heta starch at a concentration ranging from 1.5% to 3.0%. The compositions may contain the anti-CD15 antibody in a concentration ranging from 0.001 mg/L to about 15 mg/L. The composition of claim 12, wherein the concentration of Ca⁺² and Mg⁺² ions are from about 0.1 mM to about 10 mM.

Also provided are kits for separating blood tissue and concentrating the desired therapeutic cells, containing a system consisting of a cell concentration device, an effective amount of a cell separation medium and packaging material. The kits can include blood or bone marrow or blood tissues collection equipment, including but not limited to needles, vacuum tubes, or other suitable equipment for this purpose.

In some embodiments, the method for separating cells from a blood cell-containing sample comprises contacting a blood cell-containing sample with an effective amount of a cell separation medium within a sample syringe, mixing the blood cell-containing sample with the cell separation medium in the sample syringe to create a mixture, placing the syringe containing the blood cell-containing sample and the separation medium mixture in an upright position, where the plunger of the syringe is in a downward facing position, allowing said mixture to partition into an aggregate phase and a supernatant phase, opening a first 3-way valve, where the first valve is attached to the syringe containing the mixture, a filter chamber, and a first shuttle syringe, where the first valve is opened between the syringe containing the mixture and the filter chamber, opening a second 3-way valve, where the second valve is attached to the filter chamber, a second shuttle syringe, and an extraction syringe, and the second valve is opened between second shuttle syringe and the filter chamber, compressing the plunger on the syringe containing the mixture, where the supernatant phase passes into the filtration chamber through the first valve, and the aggregate phase does not pass into the filtration chamber and remains in said syringe containing the mixture, securing the aggregate within said syringe by closing the first valve at said syringe containing the mixture, opening the first valve to allow the supernatant to move between the filter chamber and the first and second shuttle syringes, moving the supernatant through the filter chamber by compressing the plungers on the first and second shuttle syringes, opening a 2-way valve, where the 2-way valve is attached to the second valve and a waste syringe, forcing the fluid contained in the supernatant through the filter chamber through the 2-way valve and into the waste syringe, extracting concentrated cells from the filter chamber with an extraction syringe, where the extraction syringe is attached to the second valve and is located between the filter and the first shuttle syringe.

Additionally, the sample used in the methods provided herein can be a human blood cell-containing sample, a peripheral blood sample, an umbilical cord sample, a bone marrow sample, disaggregated spleen tissue, disaggregated lymphatic tissue, lymphatic fluid, or menstrual fluid, or a combination thereof. The sample can be any blood cell containing fluid obtained from any organ.

In some embodiments of the methods, the cells are recovered from the supernatant phase. Also, the sample can be partitioned into the agglutinate and the supernatant phase at 1×g.

In another embodiment, an apparatus for separating blood tissue and concentrating the desired therapeutic cells is provided, and the apparatus contains a plurality of 3-way valves, one or more 2-way valves, a plurality of shuttle syringes, where each shuttle syringe contains a plunger, and where each syringe has a tip end wherein the contents of the shuttle syringe can flow out through the tip when the plunger is compressed, a sample syringe, for introducing a sample containing cells into the apparatus, where the sample syringe has a tip end wherein the contents of the sample syringe can flow out through the tip when the plunger is compressed, at least one extraction syringe, and the extraction syringe contains a plunger and a tip end, such that the contents of the syringe can flow out through the tip when the plunger is compressed, at least one waste syringe, where the waste syringe contains a plunger and a tip end, so that the contents of the syringe can flow out through the tip when the plunger is compressed, one filter chamber, where the filter chamber has a first end and a second end. In the apparatus, a first 3-way valve is attached to a first shuttle syringe at the syringe tip, a sample syringe at the syringe tip, and the first end of a filter chamber, and where a second 3-way valve is attached to the second end of the filter chamber, a second shuttle syringe at the syringe tip, and an extraction syringe at the syringe tip, and a 2-way valve is attached a waste syringe and to the filter chamber between the second end of the filter chamber and the second 3-way valve. In some embodiments, the apparatus is a single use apparatus. Optionally, the apparatus is disposable.

### DESCRIPTION OF DRAWINGS

FIG. 1 Photograph of the cell separation and concentration system.

FIG. 2 Schematic describing the method used to separate and concentrate the desired cells; FIG. 2A depicts Step 1 of the method, where bone marrow aspirate or other blood cell tissue is obtained; FIG. 2B depicts Step 2 of the method, where the cell separation medium is extracted and mixed with the bone marrow aspirate or other blood cell tissue at a ratio of 3 parts medium to 2 parts bone marrow aspirate or other blood cell tissue; FIG. 2C depicts Step 3 of the method, where the sample syringe is attached to the cell concentration system or apparatus; FIG. 2D depicts Step 4 of the method, where the sample/medium mixture is allowed to settle for an amount of time (such as 30 minutes), after the settling period, the sample/medium mixture has partitioned into 2 layers, the lower layer containing the erythrocytes and the other undesired cells, and the upper layer containing the desired cells in suspension; FIGS. 2E & 2F depict Step 5 of the method, where the plunger of the sample syringe is compressed to load the cell suspension into the filter chamber; FIG. 2G depicts Step 6 of the method, where the plungers of the shuttle syringes are compressed to concentrate the cells in suspension, and pass the filtrate into the waste syringe; FIG. 2H depicts Step 7 of the method, where the concentrated cell suspension is extracted into the extraction or final syringe.

FIG. 3 Hematological Analysis of Bone Marrow Cells Before and After Separation and Concentration with the Formulation 1 and Formulation 2 Systems. FIG. 3A depicts unprocessed bone marrow aspirate; FIG. 3B depicts bone marrow aspirate separated and concentrated by Formulation 1 System; FIG. 3C depicts bone marrow aspirate separated and concentrated by Formulation 2 System.

FIG. 4 Analysis of Bone Marrow Processed with Formulation 2 System.
A) Hematology analysis of Bone Marrow Aspirate prior to processing.
B) Hematology analysis of Bone Marrow Aspirate after processing with the Formulation 2 system.
C) Flow cytometric analysis of CD45⁺ cells for CD14 vs Side Scatter of Bone Marrow Aspirate prior to processing with the Formulation 2 system.
D) Flow cytometric analysis of CD45⁺ cells for CD34 vs Side Scatter of Bone Marrow Aspirate prior to processing with the Formulation 2 system.
E) Flow cytometric analysis of CD45⁺ cells for CD14 vs Side Scatter of Bone Marrow Aspirate after processing with the Formulation 2 system.
F) Flow cytometric analysis of CD45⁺ cells for CD34 vs Side Scatter of Bone Marrow Aspirate prior to processing with the Formulation 2 system.

FIG. 5 Culture of MSC isolated from Bone Marrow Aspirate using cell separation Formulation 2 and concentration system.

### DETAILED DESCRIPTION

This invention relates to compositions, methods and materials for the isolation of desired cells from any type of blood tissue and the concentration of those cells to therapeutically convenient volumes in a point-of-care setting. More specifically, this invention relates to a system and method of isolating therapeutically important cells from biological samples.

Blood cells were the first tissue to be successfully transplanted, in the form of transfusion of red blood cells. Transfusions were the solution to mortality resulting from acute blood loss and have led to the establishment of blood banks worldwide that store blood cells and components for therapeutic applications.

One outcome of early efforts in regenerative medicine was the establishment of the ABO antigen specificity. The discovery of the surface antigens on human erythrocytes and their diversity of expression led to the understanding that blood units had to be screened for their antigenic expression in order to determine their appropriateness for transfusion and safety for the recipient. This information led to the commonly understood situation that type O was the universal donor due to the lack of AB cell surface antigens that would elicit an immune response and that AB was the universal recipient due to the lack of immune response to AB antigens. Type O is the lack of either A or B antigens on the erythrocyte cell surface. This information resulted in the following paradigm regarding erythrocyte units: O type erythrocytes can be transplanted into people with either A, B, or AB or O subtypes, A erythrocytes can be transplanted into either A or AB subtypes; B erythrocytes can be transplanted into either B or AB subtypes; and AB erythrocytes can only be transplanted into AB individuals.

Further transplantation studies utilizing white blood cells led to the understanding of the HLA class 1 and class 2 antigenic systems that describe the appropriateness of both hematopoietic and organ transplants into the recipient. Currently bone marrow and cord blood transplants are restricted primarily by HLA compatibility and by cellularity (as measured by total nucleated cells, TNC). In the past, ABO compatibility was not a consideration with bone marrow transplantation, despite the significant contamination of the transplants with donor erythrocytes. More recent studies have suggested that transplantation of bone marrow, peripheral blood stem cells, and cord blood units that have not been fully depleted of erythrocytes may be associated with post-transplant complications. These complications include delayed red cell engraftment (Blin, et al., Impact of Donor-Recipient Major ABO Mismatch on Allogeneic Transplantation Outcome According to Stem Cell Source, Biol Blood Marrow Transplant 16, 1315-1323, 2010), immune hemolysis (Gajewski, et al., Hemolysis of Transfused Group O Red Blood Cells in Minor ABO-Incompatible Unrelated-Donor Bone Marrow Transplants in Patients Receiving Cyclosporine Without Posttransplant Methotrexate, Blood 79, 3076-3085, 1992), fatal hemolysis (Oziel-Taieb, et al., Early and Fatal Immune Hemolysis after So-Called ‘Minor’ ABO-Incompatible Peripheral Blood Stem Cell Allotransplantation, Bone Marrow Transplantation 19, 1155-1156, 1997), acute GVHD (Barone, et al., ABO System Incompatibility and Graft Versus Host Disease (GVHD) Frequency in Bone Marrow Transplanted Patients, Blood 98, 374b, 2001 Abstract), late onset hemolysis (Petz, L, Immune Hemolysis Associated with Transplantation, Semin Hematol 42: 145-155, 2005), and delayed platelet engraftment (Tomonari, et al., Impact of ABO Incompatibility on Engraftment and Transfusion Requirement after Unrelated Cord Blood Transplantation: A Single Institute Experience in Japan, Bone Marrow Transplant 40(6), 523-528).

Blood tissues, including but not limited to peripheral blood, bone marrow, umbilical cord blood, the spleen, and lymphatics contain a wide variety of cells that have been shown to have therapeutic potential. Bone marrow and umbilical cord blood contain stem cells that are capable of completely restoring a hematopoietic system. Bone marrow and cord blood transplants are the therapy of last resort in the treatment of leukemia and other blood disorders. Transplantation of those cells into the recipient is limited by the degree of match of the HLA antigens between the donor and the recipient.

As the number of procedures accumulated over the years, the parameters associated with successful engraftment have become more evident. Successful engraftment is associated with high degree of HLA compatibility, high cellularity, CD34⁺ count, and potency (as measured by colony-forming units). Critical for success is the maximal recovery of the therapeutic cells from the donated tissue, especially in the case of umbilical cord blood as there is a limited volume and only one opportunity to collect cells. In addition to hematopoietic stem cells, other cells have been identified that have been shown to have therapeutic potential. These include T-cells and B-cells that can be used in immunotherapies, dendritic cells that can be used in cellular vaccinations, platelets as a source of growth and thrombotic factors, endothelial progenitor cells for vascular therapies, and mesenchymal and multi-lineage stem cells for orthopedic therapies, immune regulation and other regenerative therapies.

Bone marrow aspirates have been used in certain orthopedic procedures, such as spinal fusion, as an aid to speed the fusion process between adjacent vertebrae. These autologous aspirates are most often acquired from the patient in the course of the surgical procedure within the surgical suite. In the case of spinal fusion, bone marrow aspirate is a commonly used additive to the fusion site in order to promote the ossification of the bone and the orthopedic device used to join the adjacent vertebrae. Most practitioners use unprocessed bone marrow aspirates and add them directly to the sponge-like and ceramic materials that are then added to the fusion site.

Osteogenic progenitor cells, such as mesenchymal stem cells found in the bone marrow aspirate, have been demonstrated to develop bone tissue in vitro and are thought to be responsible for increased fusion rates. The cells that can develop into bone in vitro have been shown to make up a very small percentage of the cells in the aspirate. In fact, published literature suggests the incidence of mesenchymal stem cells/osteogenic progenitor cells is approximately 0.001% of nucleated cells (Hernigou, et al., Percutaneous autologous Bone-Marrow Grafting for Nonunions: Influence of the Number and Concentration of Progenitor Cells, J Bone Joint Surg Am, 87(7), 1430-1437, 2005).

Recently, several centrifuge-based technologies have been developed to harvest buffy coats with the intent of reducing the volume of the aspirate and reducing erythrocytes without significantly reducing the recovery of therapeutically important cells, including but not limited to osteogenic progenitor cells. However, these new technologies have significant drawbacks. Under the intended design of these technologies, the best possible result does not provide any enrichment of the osteogenic progenitor cells within the nucleated cell component and does not reduce hematocrit significantly. This means that the vast majority of the cells given to the patient either do not contribute to the therapeutic activity of the aspirate, or worse, may actually act against healing. Pro-inflammatory granulocytes and granulocyte progenitor cells comprise a major proportion of leukocytes transplanted in bone marrow aspirates. Studies have suggested that pro-inflammatory granulocytes can contribute to muscle damage (Toumi, et al., The inflammatory Response: Friend or Enemy for Muscle Injury, Br J Sports Med, 37(4), 284-286, 2003; Schneider, et al., Neutrophil Infiltration in Exercise-Injured Skeletal Muscle: How Do We Resolve the Controversy, Sports Med, 37(10), 837-856, 2007), suppressed bone formation and bone healing (GrØgaard, et al., The polymorphonuclear leukocyte: Has it a Role in Fracture Healing, Arch Orthop Trauma Surg, 109(5), 268-271, 1990), and wound healing (Martin, et al., Wound Healing in the PU.1 Null Mouse Tissue Repair is not Dependent on Inflammatory Cells, Curr Biol, 13(13), 1122-1128, 2003; Dovi, et al., Accelerated Wound Closure in Neutrophil-Depleted Mice, J Leukoc Biol, 73(4), 448-455, 2003).

Applicants have invented systems, apparatuses, and methods to reduce erythrocytes and inflammatory granulocytes without reduction of the stem cell component in the bone marrow aspirate, which provides an improved, effective and more concentrated therapeutic for orthopedic and other therapeutic applications.

Current methods for processing bone marrow and cord blood in order to reduce volume and deplete erythrocytes require centrifugation and result in significant losses of desired cells while at the same time producing an incomplete removal of erythrocytes and inflammatory cells. In most instances this processing occurs outside the surgical suite, in part, because of air currents created by the centrifuge disturb the dead-air space needed over the incision sites.

This system has advantages over the current technology used to process biological samples. One main advantage is the lack of a centrifuge or any equipment that requires electrical power. This advantage removes one of the problems inherent in the use of centrifugation in a surgical setting, that is, the creation of air currents that could compromise the sterility of the surgical site. Another advantage of this system would be the absence of need for electrical power. This opens up the potential of this system to be used in places where electricity may be intermittent or unavailable, such as those in field military situations. Another important advantage involves the cell separation medium. The cell separation medium is superior to the current methods in the reduction of undesirable cells from the cell concentrate. This is especially important in the use of bone marrow aspirates in the field of orthopedic applications where the presence of erythrocytes and inflammatory granulocytes has been shown to have detrimental effects. The last important advantage of this system is the ability of the user to customize the desired final volume of the cell concentrate to their specific application. Currently available technology results in a fixed final volume regardless of the final application.

Applicants have invented a non-centrifuge based system that enables volume reduction and removal of erythrocytes and pro-inflammatory granulocytic cells from blood tissues of all type, while retaining a high recovery of the stem cell component. The systems and methods described in embodiments herein provide the ability to process the bone marrow aspirate within the surgical suite, which produces a superior cell composition for surgical or other therapeutic use, as compared to the unprocessed aspirate or the same aspirate processed by the current technologies. Existing technologies do not provide these benefits, and in fact, result not only in therapeutic cell loss but retention of significant erythrocyte and granulocyte contamination.

The systems and methods described herein can be used for a variety of purposes, including but not limited to the preparation of cells for tissue culture, immunophenotypic characterization, diagnostic testing, further purification, culturing, and other therapeutic applications.

The cell separation medium provided herein can be combined with packaging material and sold as a kit. The cell separation system or apparatus provided herein can be combined with packaging material and sold as a kit. The cell separation medium and the cell separation system or apparatus can be packaged together and sold as a kit. In some embodiments, the packaging material includes blood or blood tissue collection materials and equipment, including, but not limited to vacuum tubes, needles, lances, blood bags, and other suitable equipment. The kits provided herein can be single use, and disposable. The packaging material included in a kit typically contains instructions or a label describing how the components of the kit can be used to separate and concentrate the desired cells. Components and methods for producing such kits are well known.

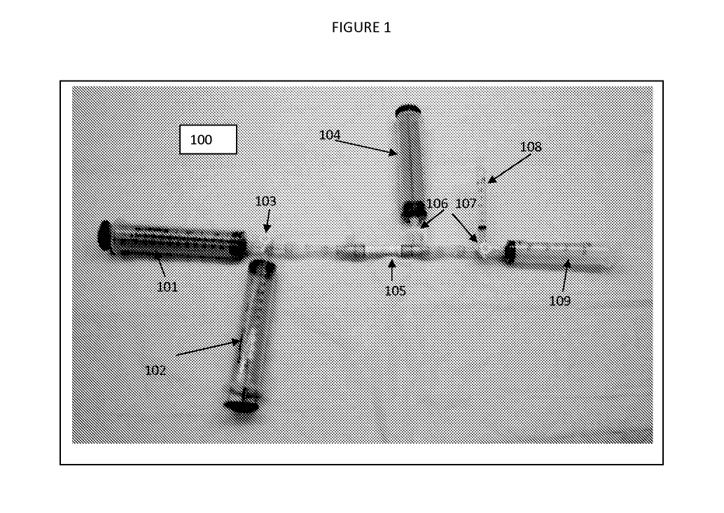

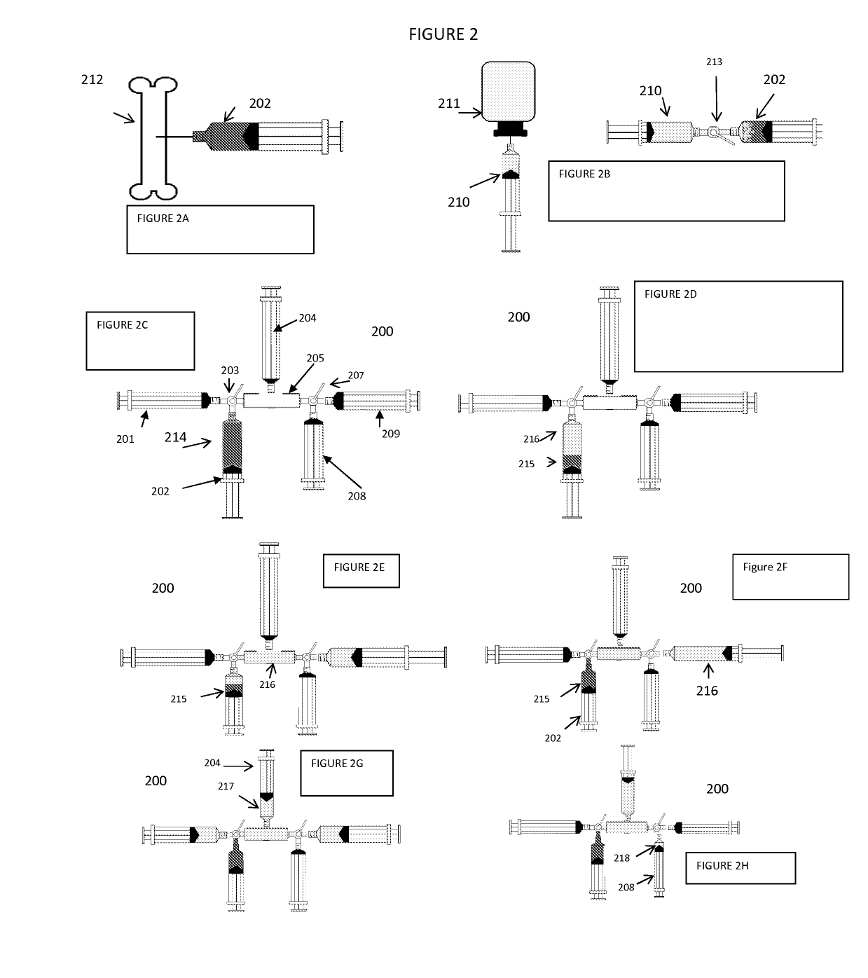

The systems and methods described herein are embodiments of Applicants' invention for the preparation of cells for tissue culture, immunophenotypic characterization, diagnostic testing, further purification, culturing, and other therapeutic applications. The systems are comprised of a series of an interconnected plurality of syringes, valves, one or more filters and one or more cell separation medium. One embodiment of Applicants' cell concentration system is shown in FIG. 1. One embodiment of Applicants' method provides the separation of a biological sample into desired cells and undesired cells, and allows for easy and simple separation and further concentration of the desired cells, as well as the reduction of volume of the bone marrow aspirate. One embodiment of the method of this system is described in FIG. 2. The methods and systems of the invention provide a complete system. No additional equipment or power source (such as electricity) is required or needed. The methods and systems of the invention produce highly concentrated cell populations with high recovery of the desired cells in a device that maintains the sterility of the cells. The methods and systems provided herein are particularly novel due to the ability to use these methods and systems to process tissue samples in areas that may be lacking in electricity or in non-aseptic conditions, including but not limited to conditions found in front line military situations, natural or other disaster areas, and impoverished or remote areas.

The biological sample used in the systems and methods provided herein can be any sample obtained from a body, including but not limited to cells from peripheral blood, umbilical cord blood, bone marrow, surgical blood recoveries, lymph fluids, lymph nodes, spleen, menstrual blood, or other organs. As used herein, blood tissue refers to cells and plasma.

In an embodiment, the concentration portion of the system provides a series of an interconnected plurality of syringes, valves, and a tangential flow filter. In some embodiments, one syringe (the sample syringe) introduces the biological sample to the concentration system, another syringe is the waste syringe that captures the liquid waste of the concentration system, two other syringes are the shuttle syringes which are used to pass the cell suspension through the filter mechanism and use pressure from both syringes to push the liquid phase of the cell suspension through the filter and out of the system and into the waste syringe. A final syringe extracts the final cell concentrate from the system for further applications. It is during this final concentration period using the shuttle syringes that the final volume of the cell suspension is determined by the user. Using the demarcations on the syringes, users can determine the fluid volume remaining in their cell suspension and customize it to their specific needs.

In an embodiment, the filter chamber is a tangential flow filter. Exemplary tangential flow filtration filters include, but are not limited to, Spectrum Labs MicroKros® and MidiKros® hollow fiber membranes, Millipore Ultracel PLC®, Pall Microza® hollow fiber systems and other suitable filters.

In another embodiment, the cell separation medium is designed to remove only erythrocytes and maximize the recovery and concentration of nucleated white blood cells and platelets. This recoverable cell population is especially important in the case of cord blood processing, where recovery of all nucleated cells is a primary concern because usability of cord blood units is often dependent upon total nucleated cellularity. Erythrocytes have a natural repulsion due to their highly negatively charged cell membranes. In this and other embodiments, the cell separation medium can be composed of substances that reduce erythrocyte zeta-potential (net negative charge on erythrocyte cell membrane) and substances that chelate Ca⁺² and Mg⁺² ions in an isotonic buffered saline solution. When mixed with the cell containing sample, the natural repulsion of the erythrocytes in the sample is neutralized and the erythrocytes form structures resembling stacked coins called “rouleaux.” These structures have a high sedimentation rate in comparison to single cells in suspension. The aggregated cells quickly settle, falling to the bottom of the container, while the single cells remain up in the liquid suspension. In certain examples of using formulation 1, the cells recovered include all varieties of nucleated leukocytes and platelets. The cell concentrate was also depleted of ˜99% of the erythrocytes, reducing the hematocrit to less than 1%.

In an embodiment, the zeta potential reducing agent is Heta starch. The concentration of the Heta starch can be about 1-5%. In an embodiment of the system, the Ca⁺² chelator can be EDTA (ethylenediaminetetraacetic acid). Other suitable Ca⁺² chelators include, but are not limited to EGTA and citrate. The concentration of EDTA can be about 0.1 mM to 50 mM.

In an embodiment of the system, the cell separation medium can be in a ratio of medium to blood tissue sample of about 1:2 to 10:1. Optimum concentrations may depend upon the individual application. In some embodiments, the range of 1:1 to 2:1 produces the high yields of desired cells and excellent removal of undesired cells. In other embodiments, other ranges produce high yields of desired cells and excellent removal of undesired cells.

In an embodiment of the system, the cell separation medium is designed to remove erythrocytes and pro-inflammatory granulocytes and monocytes. The cell separation medium can be composed of substances that reduce erythrocyte zeta-potential (negative charge on erythrocyte cell membrane), and include one or more sources of Ca⁺² or Mg⁺² ions, and an antibody directed against CD15 antigens on the surface of granulocytes in an isotonic saline solution. During the time of mixing the sample with the medium in the system, the antibody binds to the CD15 molecules on the cell surface of the granulocytes. The antibody binding activates the granulocytes and stimulates the expression of a variety of adhesion molecules such as LFA-1 (Lymphocyte Function-Associated Antigen-1, CD11a/CD18) and ICAM-1 (Intercellular Adhesion Molecule-1, CD54) that mediate the binding of granulocytes to cells expressing their binding partner, including other granulocytes and monocytes. Suitable anti-CD15 antibodies can be chosen by their non-reactivity to monocytes. Concentrations of anti-CD15 antibodies can range from 0.01 to 15 mg/L (e.g., 0.1 to 15, 0.1 to 10, 1 to 5, or 1 mg/L). Exemplary monoclonal anti-CD15 antibodies include, without limitation, AHN1.1 (Murine IgM Isotype), FMC-10 (Murine IgM Isotype), BU-28 (Murine IgM Isotype), MEM-157 (Murine IgM Isotype), MEM-158 (Murine IgM Isotype), MEM-167 (Murine IgM Isotype), and 324.3.B9 (murine IgM isotype, BioE, St. Paul, Minn.). See e.g., Leukocyte typing IV (1989); Leukocyte typing II (1984); Leukocyte typing VI (1995); Solter D. et al., Proc. Natl. Acad. Sci. USA 75:5565 (1978); Kannagi, R. et al., J. Biol. Chem. 257:14865 (1982); Magnani, J. L. et al., Archives of Biochemistry and Biophysics 233:501 (1984); Eggens, I. et al., J. Biol. Chem. 264:9476 (1989).

Cell separation compositions also can contain divalent cations (e.g., Ca⁺² and Mg⁺²). Divalent cations can be provided, for example, by a balanced salt solution (e.g., Hank's balanced salt solution) or other suitable reagents for providing divalent cations. Divalent cations are important co-factors for selectin-mediated and integrin-mediated cell-to-cell adherence. These aggregated leukocytes form large aggregates and like the aggregated erythrocytes sediment at a far faster rate than the un-aggregated cells in suspension. The resultant cell suspension is significantly reduced in erythrocytes, granulocytes and monocytes, while retaining a high recovery of lymphocytes and stem cells. These cell populations are especially important in both immune and regenerative cell therapy. The lymphocyte population is composed of T-cells, NK cells and B-cells. Each of these cell populations has an important role in the development of future immune therapies. Stem cell components of these samples, especially in the case of bone marrow aspirates and cord blood, are useful in the area of hematopoietic reconstitution via CD34⁺ hematopoietic stem cells and in the area of regenerative non-hematopoietic medicine via mesenchymal stem cells, Multilineage Progenitor Cells (U.S. Pat. Nos. 7,622,108, 7,670,596, 7,727,763, and 7,875,543) (van de Ven et al., The Potential of Umbilical Cord Blood multipotent stem cells for Nonhematopoietic Tissue and Cell Regeneration, Exp Hematol 35: 1753-1765, 2007, Berger, et al., Differentiation of Umbilical Cord Blood-Derived Multilineage Progenitor Cells into Resiratory Epithelial Cells, Cytotherapy 8(5): 480-487, 2006), endothelial progenitors cells and other cells.

In an embodiment of the system, the sample is introduced to a cell separation medium within a syringe device (sample syringe). The sample is mixed with the cell separation medium for a specified period of time. After the appropriate period of mixing, the sample containing syringe is placed in an upright position, with the plunger side of the syringe facing down.

During the mixing period, cells that are intended to be removed aggregate into homologous and heterologous cell aggregates. These aggregates have greatly accelerated sedimentation rates, causing the aggregated cells to sediment much more quickly than the unaffected non-aggregated cells. Because the aggregated cells settle quickly during the settling period, the non-aggregated cells are left in suspension in the medium above the sedimenting cells.

At the completion of sedimentation time, the erythrocytes (in the case of formulation 1) or erythrocytes, monocytes, and granulocytes (in the case of formulation 2) will have settled to the bottom of the syringe forming a well delineated demarcation between the lower level un-desired cells and the upper level containing the desired cells in suspension. At this time, the valve connecting the sample syringe to the filter chamber is opened as is the valve between the filter chamber and one of the shuttle syringes. The plunger of the sample syringe with the settled sample is compressed to push the desired cell containing suspension into the filter chamber. When the erythrocyte layer reaches the valve, the valve is closed to the sample containing syringe, which prevents erythrocytes from entering the filter chamber. At the same time, the other valve is opened to the other shuttle syringe, enabling the fluid volume from the sample to enter into both the filter chamber and the shuttle syringes. Once the fluid contents of the sample syringe are transferred to the filter chamber, the valve is employed to close off access to the sample syringe. After this point, the shuttle syringes act to keep the cells in motion across the surface of the membrane of the filtration chamber preventing them from adhering to the membrane and reducing the recovery of cells post-concentration. The valve from the filter chamber to the waste syringe is opened allowing the fluid from the cell suspension to flow into the waste syringe. As the cell suspension is shuttled through the filter chamber, light pressure is applied to both shuttle plungers forcing the liquid portion of the cell suspension to slowly flow into the waste syringe. This is continued until the fluid portion is reduced to the final desired volume. After the final volume is achieved, the cell suspension is extracted into a final syringe that can be used for injection in regenerative therapies.

As used herein, the term “syringe” refers to an instrument (as for the injection of medicine or the withdrawal of bodily fluids) that consists of a hollow barrel fitted with a plunger and a narrowed opening at one end that can optionally be fitted with a hollow needle.

An embodiment comprises a cell separation and concentration system or apparatus 100, having a shuttle syringe 101, attached to a valve 103, wherein the value 103 is also attached to a sample syringe 102, and a tangential flow filter 105, wherein the tangential flow filter is attached at one end to valve 103 and at the other end to valve 107, the tangential flow filter is also attached by valve 106 to waste syringe 104, an extraction syringe 108 is attached to the system at valve 107, and a shuttle syringe 109 is attached to the system at valve 107. One example of this embodiment is depicted in FIG. 1.

An example of an embodiment of the method of the cell separation and concentration system or apparatus 200 is depicted in FIG. 2. In FIG. 2A, sample syringe 202 is shown extracting a source of bone marrow or blood tissue 212. In FIG. 2B, a syringe 210 is drawing an amount of cell separation medium 211 (description of same provided herein) into the body of the syringe. FIG. 2B also depicts the syringe containing the cell separation medium 210 joined by a two-way valve 213, where the valve 213 is in an open position to allow the cell separation medium to flow into the sample syringe 202, which is attached to the valve 213. In FIG. 2C, an embodiment is depicted, which comprises a method of a cell separation and concentration system or apparatus 200, having a shuttle syringe 201, attached to a valve 203, wherein the value 203 is also attached to a sample syringe 202, and a tangential flow filter 205, wherein the tangential flow filter is attached at one end to valve 203 and at the other end to valve 207, the tangential flow filter is also attached by valve 206 (not shown) to waste syringe 204, an extraction syringe 208 is attached to the system at valve 207, and a shuttle syringe 209 is attached to the system at valve 207. FIG. 2C shows the mixture of blood tissue or bone marrow and cell separation medium 214 in the sample syringe 202. FIG. 2D depicts the system 200, and the sample syringe 202 shows the separation of the undesired cells 215 in the bottom of the syringe and the desired cells 216 in the top of the syringe. FIG. 2E depicts the system 200, with the undesired cells 215 in the sample syringe 202, and the desired cells 216 in the filter 205. FIG. 2F depicts the system 200, and the undesired cells 215 remaining in the sample syringe 202. FIG. 2G depicts the system 200, and the waste 217 in the waste syringe 204. FIG. 2H depicts the system 200, and the cell suspension 218 in the extraction syringe 208.

The invention is further described in the following examples, which do not limit the scope of the invention described in the claims.

### EXAMPLES

### Example 1

TABLE 1
Formula 1 for Erythrocyte Removal

| | | |
|---|---|---|
| Heta Starch | 20 | g/L |
| Phosphate Buffered Saline | 1 | L |
| EDTA | 1 | mM |

### Example 2

TABLE 2
Formula 2 for Erythrocyte, Granulocyte and Monocyte Removal

| | | |
|---|---|---|
| Heta Starch | 20 | g/L |
| Hank's buffered saline solution (10X) | 100 | mL/L |
| Anti-human CD15 (murine IgM monoclonal | 2 | mg/L |
| antibody clone 324.3.B9) | | |

Note this formula was mixed in deionized water.

### Example 3

### Filtration Chamber

The filtration chamber is a tangential flow filter. The filtration chamber has 3 ports; two ports allow the addition of fluids to the filter unit, and the third port allows the removal of the filtrate. The filtration unit is composed of a series of defined pore size tubes within a larger chamber (hollow fiber). The fluid to be concentrated is inserted into the tubes. Pressure placed on both sides of the filtration unit forces the fluid through the pores in the tubes and into the larger chamber surrounding the tubes. The filtrate is then removed from the larger chamber by extraction out the third port. By keeping the cells in suspension in motion within the tangential filter, the cells avoid getting trapped on the filter, and recovery of the cells is maximized. In this example the filtration unit used was Spectrum Laboratories X2-MO5E-100-F2N.

### Example 4

### Erythrocyte Removal

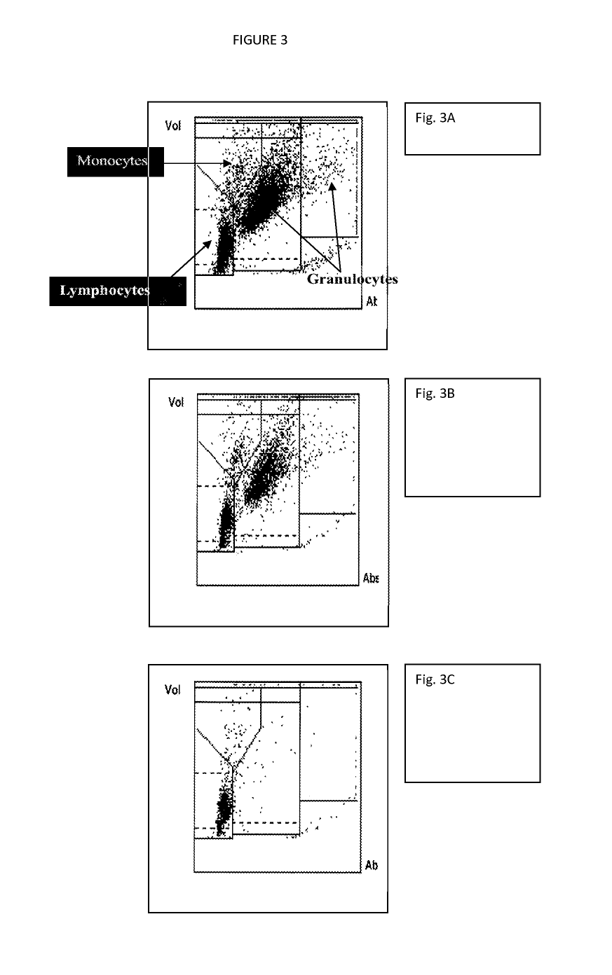

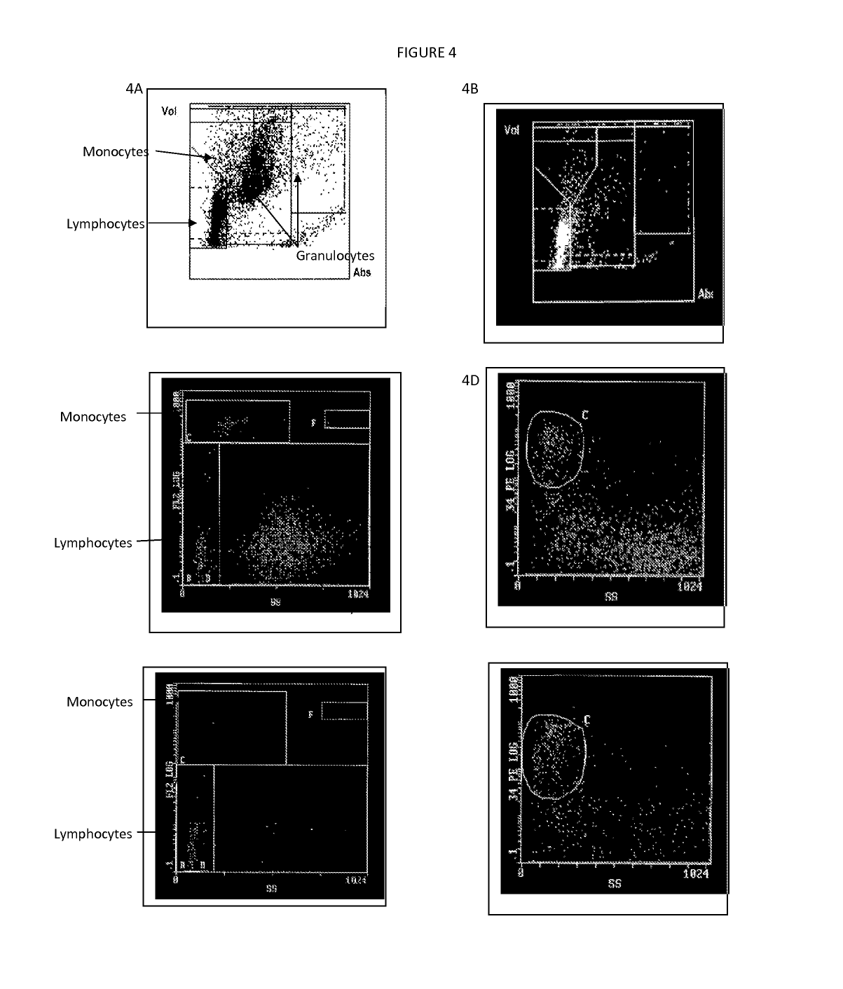

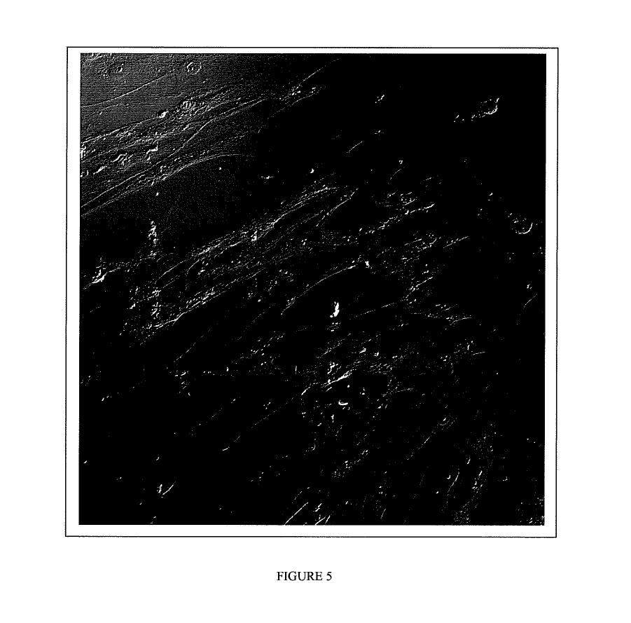

In the case of removal of erythrocytes while concentrating leukocytes and platelets, Formula 1 described in Example 1 is used as the cell separation medium. The cell separation medium is mixed with the blood sample at a ratio of 7 parts medium to 5 parts blood sample. The medium and sample are mixed for 1 minute prior to placing the sample syringe in an upright position (plunger facing down) for 30 minutes. During the 30 minutes time, erythrocytes form large aggregates and sediment quickly. The un-aggregated cells are displaced upward by the sedimenting erythrocytes and become concentrated in the supernatant above. The resultant un-aggregated cell suspension is transferred to the filtration chamber, where it is concentrated to a desired final volume using pressure from the shuttle syringes to push the fluid from the cell suspension into the waste syringe. The final cell concentrate is removed from the cell concentration system by the extraction syringe. Samples from before separation were compared to samples taken after separation, and analyzed using the Beckman Coulter AcT 5diff CP hematology analyzer. Exemplary hematology histograms from before and after separation is shown in FIG. 3A. The recoveries of leukocytes and platelets and depletions of erythrocytes of 6 different samples of peripheral blood processed by the Formula 1 cell separation and concentration system are shown below.

TABLE 3

| | Leukocyte | Platelet | Erythrocyte |
|---|---|---|---|
| Sample | Recovery | Recovery | Depletion |
| 1 | 98.6% | 99% | 99.1% |
| 2 | 91.9% | 90.8% | 98.7% |
| 3 | 93.6% | 91.5% | 98.6% |
| 4 | 91.3% | 93.2% | 98.8% |
| 5 | 97.5% | 94.5% | 97.5% |
| 6 | 90% | 95.5% | 98.5% |
| mean ± stdev | 93.8% ± 3.49 | 94.08% ± 2.98 | 98.5% ± 0.5 |

### Example 5

### Erythrocyte, Granulocyte and Monocyte Removal

In the case of removal of erythrocytes, granulocytes and monocytes while concentrating lymphocytes, stem cells and platelets, Formula 2 described in Example 2 is used as the cell separation medium. The cell separation medium is mixed with the blood sample at a ratio of 3 parts medium to 2 parts blood sample. The medium and sample are mixed for 30 minutes prior to placing the sample syringe in an upright position (plunger facing down) for 30 minutes. During the 30 minutes time, erythrocytes form large aggregates, as do granulocytes and monocytes, and the aggregates sediment quickly. The un-aggregated cells are displaced upward by the sedimenting aggregates and become concentrated in the supernatant above. The resultant un-aggregated cell suspension is transferred to the filtration chamber where it is concentrated to a desired final volume using pressure from the shuttle syringes to push the fluid from the cell suspension into the waste syringe. The final cell concentrate is removed from the cell concentration system by the extraction syringe. Samples from before separation were compared to samples taken after separation analyzed by the Beckman Coulter AcT 5diff CP hematology analyzer and by flow cytometric analysis using the Coulter Epics XL flow cytometer. Analysis of a bone marrow aspirate processed by this system by hematological and flow cytometry analysis id shown in FIG. 4. The recovery of desired cells and removal of undesired cells is shown in Table 5 below.

TABLE 5
Recovery of Desired Cells and Removal of Undesired Cells

| | | | Post-processing | | | |
|---|---|---|---|---|---|---|
| | | | | | | % |
| Pre-processing | | | | | % | De- |
| | Absolute | | Absolute | | Recov- | ple- |
| Cell Type | Number | % | Number | % | ery | tion |
| Erythrocytes | 1.85 × 10¹⁰ | — | 1.64 × 10⁸ | — | — | 99.1 |
| Leukocytes | 7.76 × 10⁷ | — | 1.64 × 10⁷ | — | 21.1 | 78.9 |
| Platelets | 2.0 × 10⁸ | — | 8.2 × 10⁷ | — | 41 | 59 |
| Lymphocytes | 1.06 × 10⁷ | 13.6 | 1.23 × 10⁷ | 75.1 | 116 | — |
| Monocytes | 2.96 × 10⁶ | 3.81 | 1.64 × 10⁵ | 1.0 | 5.5 | 94.5 |
| Granulocytes | 6.38 × 10⁷ | 82.2 | 3.87 × 10⁶ | 23.6 | 6.1 | 93.9 |
| CD3 (T cells) | 5.82 × 10⁶ | 7.5 | 8.05 × 10⁶ | 49.1 | 138 | — |
| CD34 (HSC) | 1.26 × 10⁶ | 1.63 | 1.17 × 10⁶ | 7.14 | 92.6 | — |

### Example 6

This data below shows the results of the removal of RBC and recovery of WBC and Platelets after separation with formula one reagent.

TABLE 6

| BMA | RBC Removal | WBC Recovery | PLT Recovery |
|---|---|---|---|
| 4549 | 98.5 | 95.4 | 58.74 |
| 4450 | 98.3 | 96 | 74.82 |
| 4551 | 98.6 | 98.3 | 57.04 |
| 4552 | 97.8 | 98.4 | 60.65 |
| 4553 | 98 | 99.7 | 66.14 |
| 4554 | 98.1 | 85.5 | 99.14 |
| 4574 | 97.6 | 95.1 | 50.32 |
| Ave | 98.12857143 | 95.48571429 | 66.69285714 |
| Std Dev | 0.363841933 | 4.731253735 | 16.22850036 |

The Table below (Table 7) shows the recovery of WBC after the concentration of the supernatants by the filtration device after the separation shown in Table 6.

TABLE 7

| BMA | WBC Recovery |
|---|---|
| 4549 | 91.5 |
| 4450 | 88.6 |
| 4551 | 96.5 |
| 4552 | 100.2 |
| 4553 | 99.5 |
| 4554 | 104.9 |
| 4574 | 100.7 |
| Ave | 97.41428571 |
| Std Dev | 5.663458476 |

The specific reagents and proportions are for illustrative purposes. Reagents may be exchanged for suitable equivalents and proportions may be varied, according to the desired properties of the form of interest or use.

### OTHER EMBODIMENTS

It is to be understood that while the invention has been described in conjunction with the detailed description thereof, the foregoing description is intended to illustrate and not limit the scope of the invention, which is defined by the scope of the appended claims. Changes and modifications can be made in accordance with ordinary skill in the art without departing from the invention in its broader aspects as defined in the following claims. Other aspects, advantages, and modifications are within the following claims. All publications, patents, and patent documents are incorporated by reference herein, as though individually incorporated by reference.

1-26. (canceled)

27. A non-centrifuge based system for separating blood tissue and concentrating the desired therapeutic cells comprising
(a) a cell concentration device, comprising one or more syringes, one or more valves, and a filtration device, and
(b) an effective amount of a cell separation medium.

28. The system of claim 27, wherein the one or more syringes, the one or more valves and the filtration device are assembled together to allow for the concentration of desired therapeutic cells from said blood tissue.

29. The system of claim 27, wherein the filtration device is a tangential flow filtration device.

30. The system of claim 27, wherein the cell separation medium comprises an effective amount of a zeta potential reducing agent and an effective amount of a Ca⁺² chelating agent in a buffered solution.

31. The system of claim 30, wherein the zeta potential reducing agent is Heta starch, the Ca⁺² chelating agent is EDTA, and the buffered solution is a phosphate buffered solution.

32. The system of claim 31, wherein the concentration of Heta starch is from about 1.0% to 4.0%.

33. The system of claim 31, wherein the concentration of EDTA is from about 0.05 mM to about 20 mM.

34. The system of claim 27, wherein said blood tissue is a human blood cell-containing sample, a peripheral blood sample, an umbilical cord sample, a bone marrow sample, a disaggregated spleen tissue sample, a disaggregated lymphatic tissue sample, a lymphatic fluid sample, or a menstrual fluid sample, or a combination thereof.

35. A cell separation medium for the removal of erythrocytes, granulocytes and monocytes from a blood cell containing sample, comprising:
a. an effective amount of a zeta potential reducing agent,
b. an effective amount of Ca⁺² ions,
c. an effective amount of Mg⁺² ions,
d. an effective amount of an anti-CD15 antibody,
wherein the zeta potential reducing agent, the Ca⁺² ions, the Mg⁺² ions, and the anti-CD15 antibody are contained in a buffered physiologic saline solution.

36. The medium of claim 35, wherein the concentration of Heta starch is from about 1.5% to 3.0%.

37. The medium of claim 35, wherein the concentration of the anti-CD15 antibody is from about 0.001 mg/L to about 15 mg/L.

38. The medium of claim 35, wherein the concentration of Ca⁺² and Mg⁺² ions are from about 0.1 mM to about 10 mM.

39. A non-centrifuge based apparatus for separating blood tissue and concentrating the desired therapeutic cells, comprising
a. a plurality of 3-way valves,
b. a plurality of 2-way valves,
c. a plurality of shuttle syringes;
d. a sample syringe;
e. at least one extraction syringe;
f. at least one waste syringe;
g. at least one filter chamber, wherein each filter chamber has a first end and a second end,
and wherein each syringe contains a plunger and has a tip end.

40. The apparatus of claim 39, further comprising
a. a first 3-way valve, wherein said first 3-way valve is attached to a first shuttle syringe at the syringe tip, a sample syringe at the syringe tip, and the first end of a filter chamber;
b. a second 3-way valve, wherein said second 3-way valve is attached to the second end of the filter chamber, a second shuttle syringe at the syringe tip, and an extraction syringe at the syringe tip; and
c. a 2-way valve, wherein said 2-way valve is attached a waste syringe and to the filter chamber between the second end of the filter chamber and the second 3-way valve.

41. (canceled)

42. The apparatus of claim 39, wherein said blood tissue is a human blood cell-containing sample, a peripheral blood sample, an umbilical cord sample, a bone marrow sample, a disaggregated spleen tissue sample, a disaggregated lymphatic tissue sample, a lymphatic fluid sample, or a menstrual fluid sample, or a combination thereof.

43. The apparatus of claim 39, wherein the apparatus is a single use apparatus.

44. A non-centrifuge based method for separating cells from a blood cell-containing sample, said method comprising:
a. mixing a volume of a blood cell-containing sample with an effective amount of a cell separation medium in the sample syringe of the apparatus of claim 39 to create a mixture;
b. placing the syringe containing the mixture in an upright position, wherein the plunger of the syringe is in a downward facing position, allowing said mixture to partition into an aggregate phase and a supernatant phase;
c. opening a first 3-way valve, wherein the first valve is attached to the syringe containing the mixture, a filter chamber, and a first shuttle syringe, wherein the first valve is opened between the syringe containing the mixture and the filter chamber;
d. opening a second 3-way valve, wherein the second valve is attached to the filter chamber, a second shuttle syringe, and an extraction syringe, and the second valve is opened between second shuttle syringe and the filter chamber;
e. compressing the plunger on the syringe containing the mixture, wherein the supernatant phase passes into the filtration chamber through the first valve, and the aggregate phase does not pass into the filtration chamber and remains in the syringe that contained the mixture;
f. securing the aggregate within said syringe by closing the first valve at said syringe that contained the mixture;
g. opening the first valve to allow the supernatant to move between the filter chamber and the first and second shuttle syringes;
h. moving the supernatant through the filter chamber by compressing the plungers on the first and second shuttle syringes;
i. opening a 2-way valve, wherein the 2-way valve is attached to the second valve and a waste syringe;
j. forcing the fluid contained in the supernatant through the filter chamber through the 2-way valve and into the waste syringe;
k. extracting concentrated cells from the filter chamber in the volume desired with an extraction syringe, wherein the extraction syringe is attached to the second valve and is located between the filter and the first shuttle syringe.

45. The method of claim 44, wherein said sample is any blood cell containing sample is a human blood cell-containing sample, a peripheral blood sample, an umbilical cord sample, a bone marrow sample, a disaggregated spleen tissue sample, a disaggregated lymphatic tissue sample, a lymphatic fluid sample, or a menstrual fluid sample, or a combination thereof.

46. (canceled)

47. The apparatus of claim 39, wherein the size of the sample syringe is selected based on the volume of the sample.

48. The apparatus of claim 39, wherein the extraction syringe is selected based on the volume of concentrate desired.
